(19) United States
(12) Patent Application Publication
(10) Pub. No.: US 2014/0230827 A1
(43) Pub. Date: Aug. 21, 2014

(54) Devices and Methods for Cardiac Catheterization

(71) Applicants: R. Lee Jobe, Raleigh, NC (US); Christopher Allen Gardner, Waxhaw, NC (US); Mikail Adam Mannan, Raleigh, NC (US); Ross Ivey Spence, Otto, NC (US); Ramey Brooke Williams, Cary, NC (US); Chima U. Igboko, Raleigh, NC (US)

(72) Inventors: R. Lee Jobe, Raleigh, NC (US); Christopher Allen Gardner, Waxhaw, NC (US); Mikail Adam Mannan, Raleigh, NC (US); Ross Ivey Spence, Otto, NC (US); Ramey Brooke Williams, Cary, NC (US); Chima U. Igboko, Raleigh, NC (US)

(21) Appl. No.: 14/181,101

(22) Filed: Feb. 14, 2014

Related U.S. Application Data
(60) Provisional application No. 61/765,245, filed on Feb. 15, 2013.

(51) Int. Cl. A61B 19/00; A61B 17/135; A61M 25/01

(52) U.S. Cl. CPC A61B 19/203; A61M 25/01; A61B 17/135
USPC 128/845

(57) ABSTRACT

A device for fixating and/or supporting a wrist and hand of a patient during a radial artery procedure includes a frame. The frame includes a wrist frame member having a wrist support surface to receive the wrist of the patient. The frame includes a hand frame member having a hand support surface to receive the hand of the patient. The hand support member is pivotally connected to the wrist support member. The hand frame member is movable relative to the wrist frame member between a first position to a second position. In the first position, the hand support surface is at a first angle relative to the wrist support surface to hyperextend the wrist to promote access to the artery for the procedure. In the second position, the hand support surface and the wrist support surface are generally aligned to straighten the wrist and promote hemostasis after the procedure.

### RELATED APPLICATIONS

This application claims priority from U.S. Provisional Application No. 61/765,245, filed Feb. 15, 2013, the disclosure of which is hereby incorporated herein in its entirety.

### BACKGROUND

Heart disease is the leading cause of death in the United States and attributes to approximately 600,000 deaths each year. Additionally, more than one-third of people in the U.S. are obese and therefore at a higher risk for heart disease. Cardiac catheterization is a procedure that is used to evaluate the heart's health. It is also used in treatment of heart attacks, one of the most common symptoms of heart disease, to open blocked arteries. Catheterization is usually initiated through a patient's femoral artery, but this is especially difficult for bariatric (obese) patients due to excess body fat. Therefore, there is a need to increase the ease of cardiac catheterization for bariatric patients.

For bariatric patients, cardiac catheterization through the radial artery (trans-radial catheterization) is considerably easier. It may also be advantageous over catheterization through the femoral artery due to a lower risk of bleeding at the incision site and reduced pain due to decreased time of compression at the incision site. In addition, patients are free to move around shortly after the procedure instead of having to lie flat for several hours.

Cardiac catheterization is growing in popularity among patients and cardiologists due to the significant benefits for patients in ease and speed of recovery. However, it is estimated that only about 5% of cardiologists use radial access due to two primary problems that have hindered the expansion of the use of the radial access for cardiac catheterizations: 1) difficulty in gaining access to the radial artery; 2) and a need for stopping the bleeding afterwards while maintaining the integrity of the radial artery.

### SUMMARY

According to a first aspect, embodiments of the invention are directed to a device for fixating and/or supporting a wrist and hand of a patient during a radial artery procedure. The device includes a frame, the frame comprising: a wrist frame member having a wrist support surface to receive the wrist of the patient; and a hand frame member having a hand support surface to receive the hand of the patient, wherein the hand support member is pivotally connected to the wrist support member. The hand frame member is movable relative to the wrist frame member between a first position, wherein the hand support surface is at a first angle relative to the wrist support surface to hyperextend the wrist to promote access to the artery for the procedure, and a second position, wherein the hand support surface and the wrist support surface are generally aligned to straighten the wrist and promote hemostasis after the procedure. In some embodiments, the device includes: a wrist strap attached to the wrist frame member, the wrist strap configured to overlay the wrist of the patient and urge the wrist of the patient against the wrist support surface; and a hand strap attached to the hand frame member, the hand strap configured to overlay the hand of the patient and urge the hand of the patient against the hand support surface. The wrist strap may have first and second opposed ends with at least one of the first and second ends removably attached to the wrist frame member, and the hand strap may have first and second opposed ends with at least one of the first and second ends removably attached to the hand frame member.

In some embodiments, the device includes an inflatable member strap having first and second opposed ends attached to the wrist frame member and an inflatable member disposed between the first and second ends of the inflatable member strap. The device may include a pressurized fluid source in fluid communication with the inflatable member and configured to inflate the inflatable member. The pressurized fluid source may include a pump disposed on the wrist frame member. The device may include a valve disposed between the pressurized fluid source and the inflatable member, with the valve being movable between a first position, wherein pressurized fluid from the pressurized fluid source flows to the inflatable member to inflate the member, and a second position, wherein fluid is released from the inflatable member. The valve may be disposed on the wrist frame member. The device may include an instrumentation port in fluid communication with the inflatable member, the instrumentation port configured to connect with instrumentation to monitor and/or display the pressure of the inflatable member. At least one of the first and second ends of the inflatable member strap may be releasbly attached to the wrist frame member.

In some embodiments, the inflatable member strap comprises first and second straps with the inflatable member disposed therebetween, and wherein at least one of the first and second straps is releasbly attached to the wrist frame member.

The inflatable member may be adjustable along a length of the inflatable member strap.

A bottom surface of the inflatable member may include a hemostatic biomaterial. The hemostatic biomaterial may include chitosan.

In some embodiments, a rigid guard disposed between the first and second ends of the inflatable member strap and above the inflatable member, the guard configured to urge the inflatable member against the wrist of the patient as the inflatable member is inflated.

In some embodiments, a support member may extend between a bottom surface of the wrist frame member and the hand frame member.

Each of the wrist frame member and the hand frame member may have an arcuate profile.

In some embodiments, the wrist support surface comprises a pad attached to the wrist frame member and the hand support surface comprises a pad attached to the hand frame member.

The wrist frame member and the hand frame member are pivotally connected by a pivot mechanism that is configured to lock the frame in each of the first position and the second position.

According to a second aspect, embodiments of the invention are directed to a method for performing catheterization via a radial artery and thereafter establishing hemostasis of the radial artery. The method comprises: positioning a wrist and an associated hand of a patient in a device such that the posterior aspect of the wrist rests against a support surface of a first portion of the device and such that the dorsal aspect of the hand rests against a support surface of a second portion of the device, wherein the first and second portions are movably coupled together; pivoting the second portion of the device downwardly to an angled position such that the support surface of the second portion is at an angle of between about 30 and 60 degrees relative to the support surface of the first portion and such that the wrist is hyperextended or supinated; locking the second portion in the angled position; and inserting a catheter in the radial artery of the hyperextended wrist.

In some embodiments, the method further comprises: removing the catheter from the transradial artery, thereby exposing a puncture site; pivoting the second portion of the device upwardly to a flat position such that the support surface thereof and the support surface of the first portion are generally aligned; positioning and securing an inflatable member over the puncture site; and inflating the inflatable member to a pressure sufficient to seal the radial artery. In some embodiments, the method further comprises: monitoring the pressure of the inflatable member to determine whether the artery is patent or occluded; and adjusting the pressure of the inflatable member to establish hemostasis of the radial artery.

The method may include positioning and securing a wrist strap over the anterior aspect of the wrist and positioning and securing a hand strap over the palmar aspect of the hand.

Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE FIGURES

FIG. 1 is a side perspective view of a device according to some embodiments.

FIG. 2 is a top perspective view of the device of FIG. 1.

FIGS. 3A and 3B are side views of the device of FIG. 1 in an angled position and a flat position, respectively.

FIG. 4 is a bottom perspective view of the device of FIG. 1.

FIG. 5 illustrates a patient's hand and wrist in the device of FIG. 1 with the device in the flat position.

FIG. 6 illustrates a patient's hand and wrist in the device of FIG. 1 with the device in the angled position and the patient's wrist hyperextended.

FIG. 7 illustrates a patient's hand and wrist in the device of FIG. 1 with the device in the flat position and an inflatable member overlaying a puncture site.

FIG. 8 is a graph illustrating the pressure in the inflatable member of FIG. 7 over time.

FIG. 9 is a flow chart illustrating exemplary operations according to some embodiments.

FIG. 10 is a perspective view of a monitoring device for use with the device of FIG. 1 according to some embodiments.

### DETAILED DESCRIPTION

The present invention now will be described more fully hereinafter with reference to the accompanying drawings, in which illustrative embodiments of the invention are shown. In the drawings, the relative sizes of regions or features may be exaggerated for clarity. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that when an element is referred to as being “coupled” or “connected” to another element, it can be directly coupled or connected to the other element or intervening elements may also be present In contrast, when an element is referred to as being “directly coupled” or “directly connected” to another element, there are no intervening elements present. Like numbers refer to like elements throughout.

In addition, spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly.

The terminology used herein is for the purpose of describing particular embodiments only and is not intended to be limiting of the invention, As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the expression “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It is noted that any one or more aspects or features described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

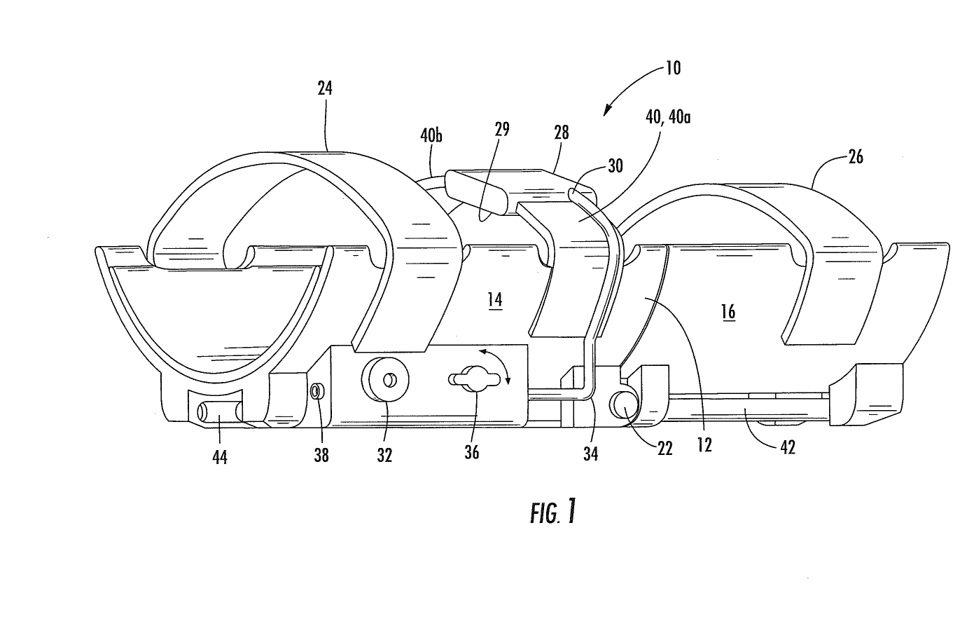

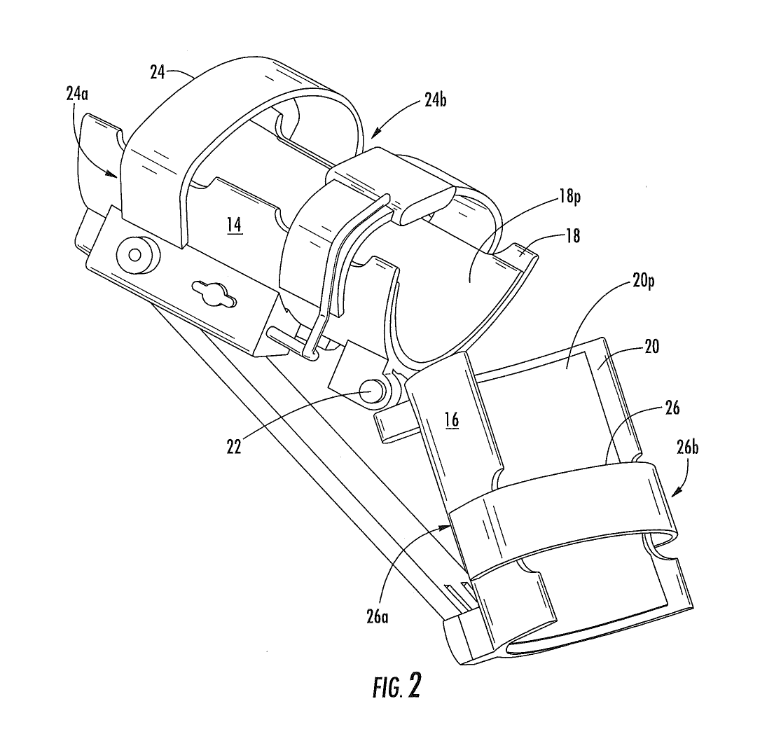

A hand and wrist fixation device 10 according to some embodiments is illustrated in FIGS. 1-4. The device 10 includes a frame 12. The frame 12 includes a forearm or wrist frame member 14 and a hand frame member 16. The forearm or wrist frame member 14 includes a forearm or wrist support surface 18 and the hand frame member 16 includes a hand support surface 20 (FIG. 2). The wrist support surface 18 may include at least one pad 18p attached to the wrist frame member 14. Similarly, the hand support surface 20 may include at least one pad 20p attached to the hand frame member 16. Each of the wrist frame member 14 and the hand frame member 16 may have an arcuate profile to conform to the shape of the patient's arm, to provide comfort and/or to restrict movement during and after a procedure. Each of the wrist frame member 14 and the hand frame member 16 may be formed of any suitable material; an exemplary material is ABS plastic. Each of the pads 18p, 20p may be formed of any suitable material; an exemplary material is chemical resistant polyethylene.

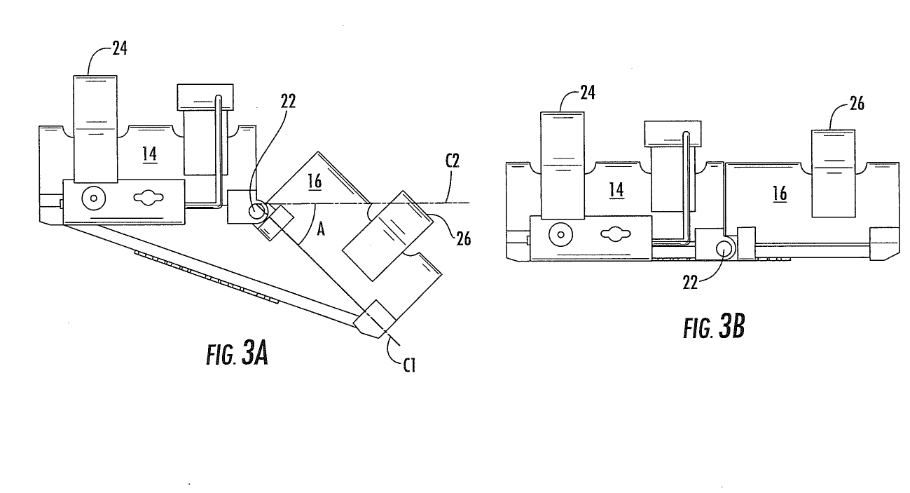

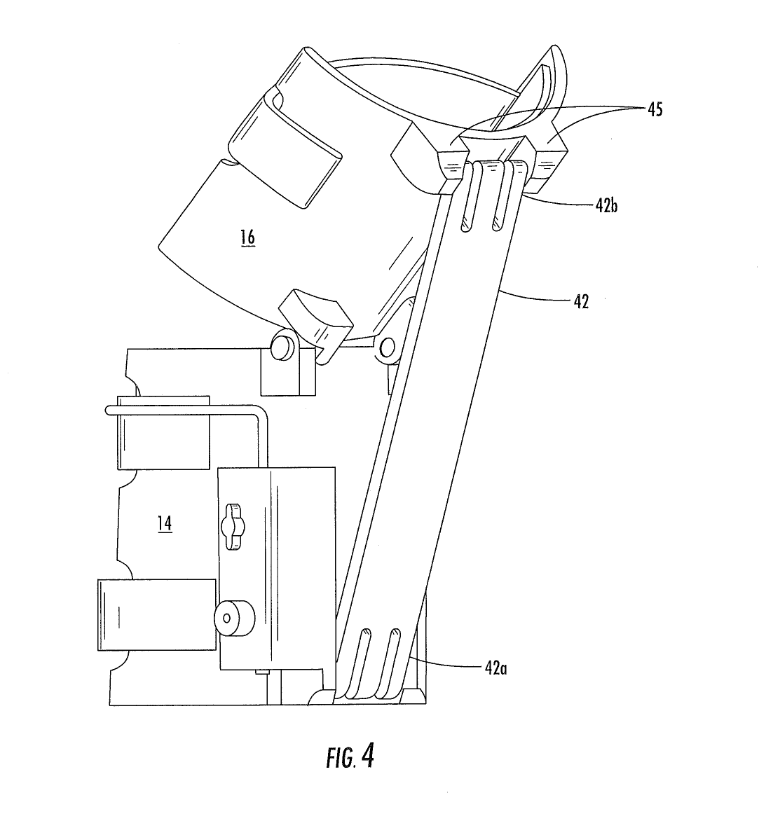

The hand frame member 16 is hingedly or pivotally connected to the wrist frame member 14 via a pivot mechanism 22. The hand frame member 16 is movable or pivotable between an angled position (FIG. 3A) and a flat position (FIG. 3B). In the angled position, the hand frame member 16 is pivoted downwardly relative to the wrist frame member 14 such that the hand support surface 20 is at a first angle A relative to the wrist support surface 18. More particularly, a longitudinal axis C1 defined by the center of the hand support surface 20 is at the angle A relative to a longitudinal axis C2 defined by the center of the wrist support surface 18.

In some embodiments, the angle A is between about 30 degrees and about 60 degrees. In some embodiments, the angle A is between about 35 and about 55 degrees. In some embodiments, the angle A is between about 40 and about 50 degrees. In some embodiments, the angle A is about 45 degrees. The hand frame member 14 is lockable in the angled position. In some embodiments, the pivot mechanism 22 may be configured to lock the hand frame member 14 in the angled position. Alternatively, a separate locking mechanism (not shown) may be provided.

In the flat position, the hand support surface 20 and the forearm support surface 18 are generally aligned. The hand frame member 14 is lockable in the flat position. In some embodiments, the pivot mechanism 22 may be configured to lock the hand frame member 14 in the flat position. Alternatively, a separate locking mechanism (not shown) may be provided.

Referring again to FIG. 1, at least one forearm or wrist strap 24 is attached to the forearm or wrist frame member 14. The wrist strap 24 is configured to overlay the anterior aspect of a patient's wrist and urge the posterior aspect of the patient's wrist against or toward the wrist support surface 18. The wrist strap 24 is adjustable to facilitate placement of the patient's wrist in the device 10 and to accommodate varying wrist sizes. Any adjustment mechanism known to those of skill in the art is contemplated. By way of example, one or both of first and second opposed ends 24a, 24b (FIG. 2) of the wrist strap 24 may be releasably attachable to the wrist frame member 14 via hook and loop fasteners. One or both of the first and second ends 24a, 24b may be released from the wrist frame member 14 while the patient's wrist is inserted therein and then reattached to the wrist frame member 14 at a position that accommodates the particular patient. By way of further example, the strap 24 may be elastic or stretchable. By way of still further example, a buckle, clasp or other device may be provided with the strap 24 to facilitate loosening and tightening of the strap 24.

At least one hand strap 26 is attached to the hand frame member 16. The hand strap 26 is configured to overlay the palmar aspect of a patient's hand and urge the dorsal aspect of the patient's hand against or toward the hand support surface 20. The hand strap 26 is adjustable to facilitate placement of the patient's hand in the device 10 and to accommodate varying hand sizes. Any adjustment mechanism known to those of ordinary skill in the art is contemplated, including those exemplary mechanisms set forth above in connection with the wrist strap 24. For example, the hand strap may have first and second opposed ends 26a, 26b (FIG. 2), and one or both may be releasably attachable to the hand frame member 16 via a suitable attachment mechanism such as hook and loop fasteners. In some embodiments, the wrist strap 24 and/or the hand strap 26 are constructed of a polymeric material (e.g., polypropylene).

The device 10 further includes an inflatable member 28 (e.g., an inflatable balloon, bladder or cuff) operatively connected to the wrist frame member 14. The inflatable member 28 includes at least one port 30 for the ingress and egress of fluid. In the illustrated embodiment, a pump 32 is disposed on the wrist frame member 14 to pressurize the inflatable member 28. The inflatable member 28 is in fluid communication with the pump 32 via a fluid flow path including tubing 34. The pump 32 may be actuated manually (e.g., by a finger, thumb or instrument) to supply pressurized fluid to the inflatable member 28. In other embodiments, the pump 32 may be actuated electronically, pneumatically, etc.

Also in the illustrated embodiment, a valve 36 is disposed on the wrist frame member 14 to depressurize the inflatable member 28. The inflatable member 28 is in fluid communication with the valve 36 via a fluid flow path including tubing 34. In some embodiments, the valve 36 may be a stopcock or the like and may be opened to open the fluid flow path between the inflatable member 28 and the pump 32. The valve 36 may be closed to seal the fluid flow path and allow pressurization of the inflatable member 28 by the pump 32.

Still referring to FIG. 1, the device 10 may also include an instrumentation port 38 for the connection of instrumentation that measures, monitors, senses and/or displays the pressure of the fluid within the inflatable member 28 and/or characteristics of blood flow through an artery, as described in greater detail below.

It will be appreciated that other configurations with respect to the pump, valve and instrumentation port are contemplated. For example, one or more of these components may be disposed away from the device 10 (i.e., not on the frame 12). Further, one or more of these components may be configured differently. For example, a different pressurized fluid source may replace the pump. The pressurized fluid source may be automated or semi-automated (e.g., not require repeated actuation).

The inflatable member 28 may be attached or connected to the wrist frame member 14 via at least one strap 40. The strap 40 may be adjustable in the same manner as the straps 24, 26 described above to accommodate patients having wrists of varying size. As will be described below, the inflatable member 28 is not used during an initial catheterization process but is placed over a puncture site after the catheterization process. As such, the inflatable member 28 will typically be displaced from the position shown in FIG. 1 until after the catheterization process is complete. In this regard, the strap 40 may have two segments 40a, 40b on opposite sides of the inflatable member 28. At least one of the segments 40a, 40b may be releasably attachable to the wrist frame member 14 to displace or remove the inflatable member 28 until the catheterization process is complete. The releasable nature of at least one of the segments 40a, 40b may be used alone or in combination with another mechanism to adjust the inflatable member 28 and the strap 40 to fit a particular patient. It is noted that the adjustable nature of the strap 40 may also facilitate proper placement of the inflatable member 28 over the puncture site at the appropriate time. Additionally or alternatively, the inflatable member 28 may be slidable or otherwise movable along a length of the strap 40 to facilitate proper placement of the inflatable member 28.

The inflatable member 28 may be clear to provide visual access for the clinician while placing the inflatable member 28 over a puncture site and/or while monitoring the puncture site for bleeding. The inflatable member 28 may be constructed of any suitable material; in some embodiments, the inflatable member 28 is constructed of thermoplastic polyurethane.

As shown in FIG. 1, an underside of the inflatable member 28 (i.e., the side in contact with the puncture site) may comprise a hemostatic biomaterial 29. For example, a chitosan-based film may be provided on the underside of the inflatable member 28, and the chitosan-based film may be infused with a proprietary ingredient that interacts with the body and the clotting cascade. Since many cardiac catheterization patients are prescribed blood thinners, inducing the clotting cascade quickly may be important. The biomaterial facilitates clotting in patients with varying level of blood thinner medications. The chitosan in the film also provides antimicrobial properties that help ward off infections at the incision site. The film may be clear to ensure that the wound site is visible and that medical providers can monitor it throughout patient recovery.

Although not illustrated, a clear rigid guard may be disposed over the inflatable member 28. For example, the rigid guard may be adhered to the top surface of the inflatable member 28 and/or connected to the strap 40.

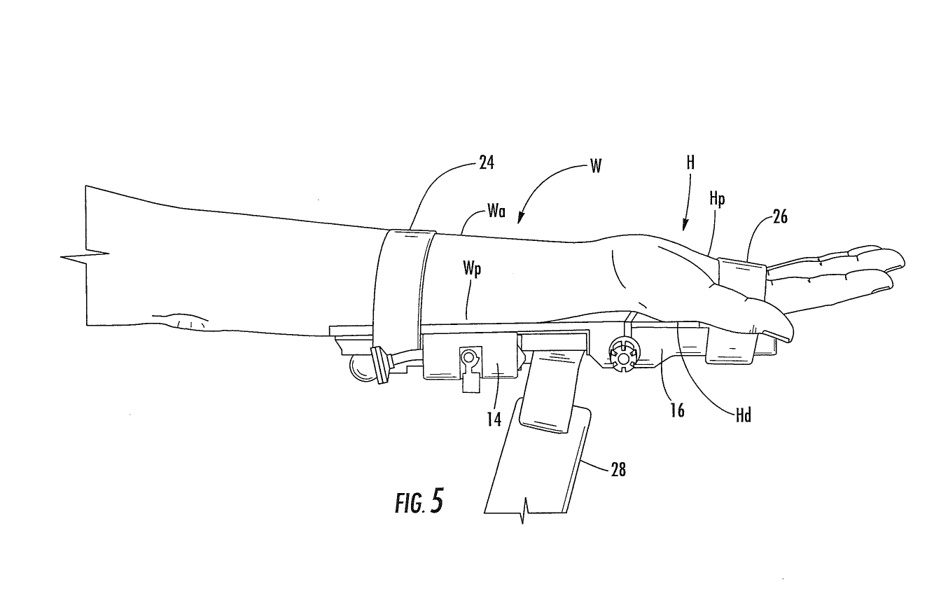

Referring to FIGS. 1 and 4, the device 10 may also include a support member 42 extending between the wrist frame member 14 and the hand frame member 16. The support member 42 includes opposite proximal and distal end portions 42a, 42b. The support member 42 may provide additional stability between the members 14, 16, including when the device is in the angled position as shown in FIG. 4. The support member proximal end portions 42a may be slidably received in a slot 44 of the wrist frame member 14. The distal end portion 42b is attached to support members 45 on the hand frame member 16. The support member 42 and/or the slot 44 may include a detent or other latching mechanism to help lock the device in the angled position and/or the flat position. The support mechanism 42 may also provide a flat surface and/or mounting structure for stabilization and/or mounting the device to a bed, table or the like.

Operation of the device will now be described with reference to FIGS. 5-7. Referring to FIG. 5, a patient's wrist W and associated hand H is positioned in the device such that the posterior aspect of the wrist Wp rests against the support surface of the wrist frame member 14 and such that the dorsal aspect of the hand Hd rests against the support surface of the hand frame member 16. The wrist strap 24 is positioned and secured over the anterior aspect of the wrist Wa. Likewise, the hand strap 26 is positioned and secured over the palmar aspect of the hand Hp. The wrist strap 24 and the hand strap 26 help to immobilize the patient's wrist W and hand H, respectively, during and after a catheterization procedure.

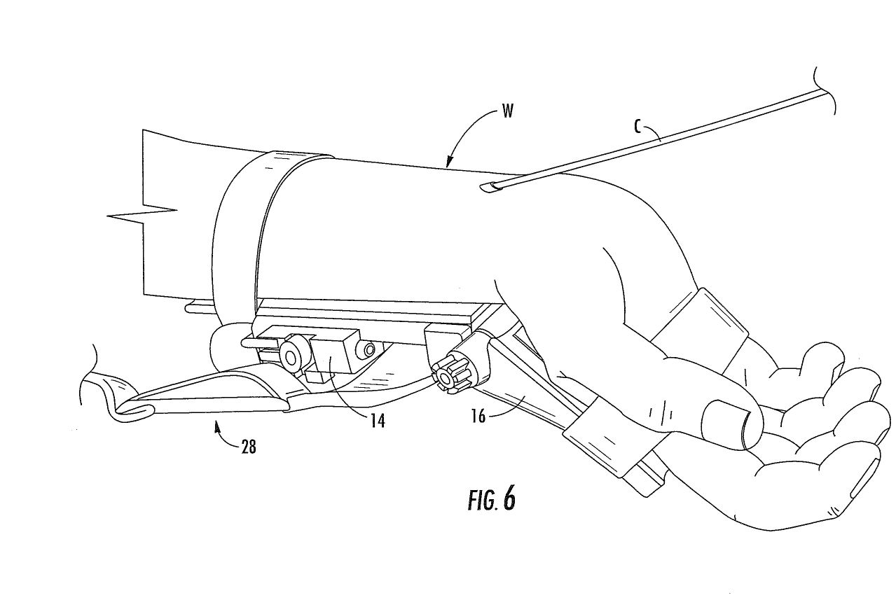

Referring to FIG. 6, the hand frame member 16 is pivoted downwardly to the angled position such that such that the wrist W is hyperextended or supinated; the hand frame member 16 is locked in the angled position using a locking mechanism, as described above. A catheter C is inserted in the transradial artery of the hyperextended wrist W to perform a transradial catheterization procedure. After the procedure, the catheter C is removed from the transradial artery, thereby exposing a puncture site. It is noted from FIGS. 5 and 6 that the inflatable member 28 is displaced during the transradial catheterization procedure.

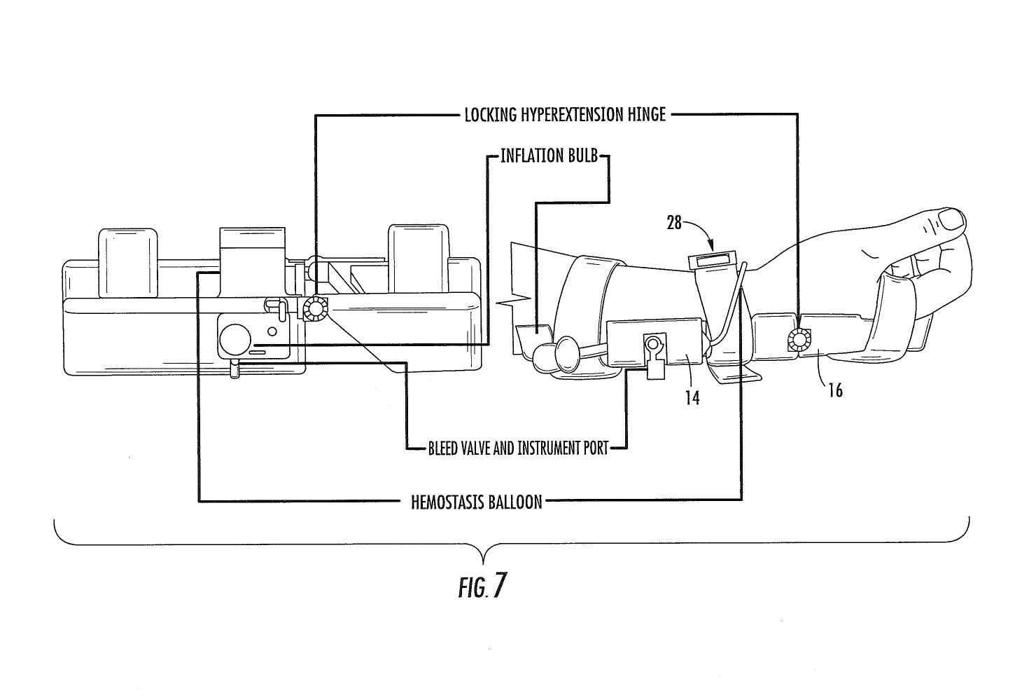

Turning now to FIG. 7, the hand frame member 16 is next pivoted upwardly to the flat position and the inflatable member 28 is positioned and secured over the puncture site. The inflatable member 28 is inflated to a pressure sufficient to seal the transradial artery. Instrumentation connected to the instrumentation port is used to monitor the pressure of the inflatable member and determine whether the transradial artery is patent or occluded. With aid of the instrumentation, the pressure of the inflatable member 28 is adjusted to establish hemostasis of the transradial artery.

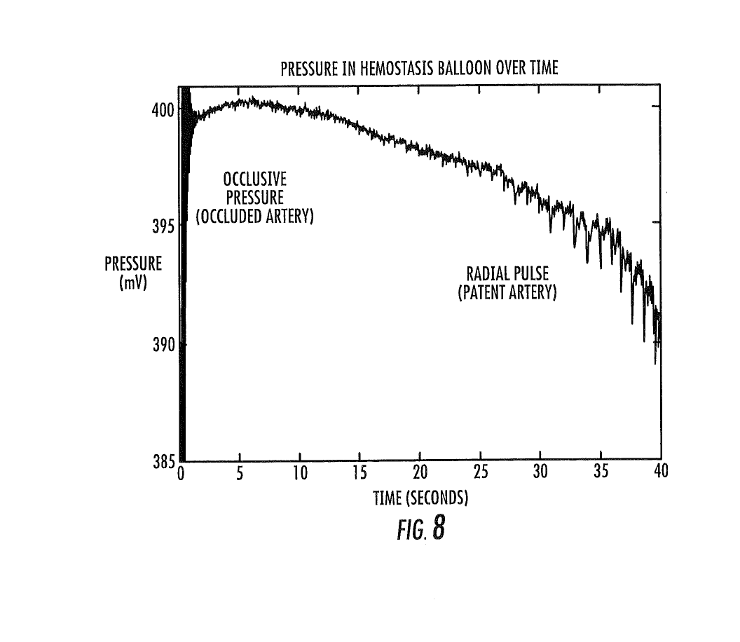

More specifically, an external pressure sensor, along with a conditioning circuit, may be attached to the outside of the device 10 to allow for the detection of the radial pulse. Detecting the radial pulse helps to ensure patent hemostasis because, if there is a pulse, the physicians know the vessel is under just enough pressure (and not too much that can occlude the vessel). The sensor may monitor the pressure as the clinician slowly relieves pressure in the vessel. Once the vessel is no longer occluded, the sensor picks up oscillations in the inflatable member 28 due to the radial pulse. At this point the clinician can lock in the pressure and the patient can leave after observation that hemostasis is achieved. FIG. 8 is an exemplary graph illustrating pressure in the inflatable member 28 over time as the pressure is relieved and stabilized.

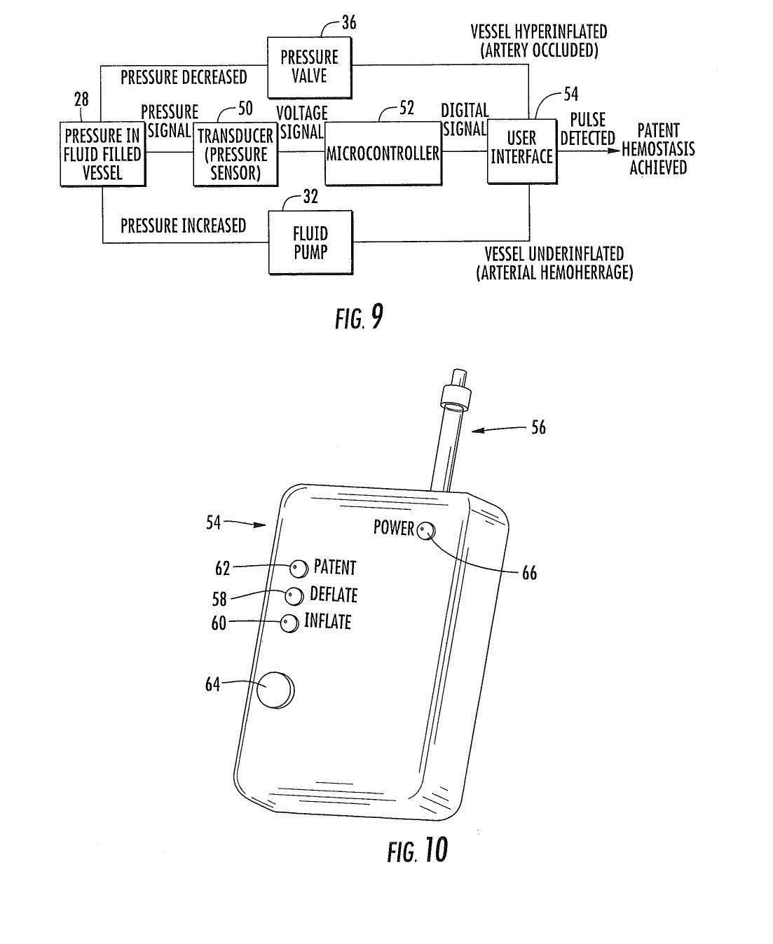

FIG. 9 is a flow chart illustrating exemplary operations according to some embodiments. FIG. 9 also illustrates various components or instrumentation that may be used with the device 10. As illustrated, a pressure sensor 50 such as a transducer receives a pressure signal from the inflatable member 28. The transducer 50 converts air pressure to voltage and a voltage signal is received at a microcontroller or microprocessor 52. The microcontroller 52 converts the analog pressure voltage to a digital signal. The microcontroller 52 also implements an algorithm that filters the digital signal and identifies the patient's pulse as the pressure is slowly reduced. At maximum pressure, the vessel will be occluded and the microcontroller will not see a periodic waveform which is the pulse. As the pressure is reduced, the microcontroller will start to detect the periodic pressure wave (i.e., the pulse) and prompt the user that the vessel is patent (e.g., with a user interface 54).

FIG. 10 illustrates an exemplary user interface 54. The user interface 54 may also be considered an external patency detection device. The external patency detection device 54 includes tubing and/or a fitting for connection to the instrumentation port 38 of the device 10 (FIG. 1). After connection, the valve 36 of the device 10 may be adjusted to allow the external patency detection device 54 to detect pressure of the inflatable member 28. The external patency detection device 54 may be turned on by, for example, actuating a power button 64 which may illuminate an LED 66. As illustrated, the external patency detection device 54 includes at least three indicators, such as LEDS, 58, 60, 62, to provide visual feedback to the user. After the inflatable member 28 is inflated (e.g., fully inflated), the “deflate” LED 58 should be illuminated. If either the “inflate” LED 60 or the “patent” LED 62 is illuminated, the inflatable member 28 should be inflated until the “deflate” LED 58 illuminates. The pressure in the inflatable member 28 is slowly released until the “patent” LED 62 illuminates; this indicates that the vessel is now patent and ready for recovery. It is contemplated that other indicators, such as dials, displays and the like, may be used instead of or in conjunction with the LEDs.

Various components such as the transducer 50 and the microcontroller 52 may be housed in the external patency detection device 54. In addition, it is contemplated that other components may be disposed in or on the external patency detection device 54. For example, the pump 32 and/or the valve 36 may be disposed on the external patency detection device 54 in some embodiments.

The device 10 described above may provide several advantages with respect to radial catheterizations. The device 10 hyperextends the wrist W in the locked angled position to provide easier access to the radial artery during a catheterization. The device 10 locks into a flat position following the procedure so that the patient's wrist is immobilized and the recovery is not compromised by accidentally bending and moving the wrist and arm. The hand and wrist straps 26, 24 also help to immobilize the patient's wrist W. The device 10 uses pneumatic compression to seal the incision site while ensuring that the radial artery does not occlude. The device 10 includes or may be used with instrumentation that detects whether or not the radial artery is patent and allows the physicians and nurses to adjust the pneumatic pressure to the ideal pressure for each individual patient. Also, a proprietary biomaterial may be incorporated onto the surface of the compression mechanism (i.e., the inflatable member 28) that helps to induce and speed up hemostasis by inducing the natural clotting cascade, as well as mitigate the risk of infection at the radial entry site due to the antimicrobial properties.

The device 10 may provide additional advantages. The device is a comprehensive hemostatic device for use with trans-radial catheterizations. Trans-radial catheterization is an innovative vascular access method used in a variety of procedures to diagnose and treat vascular disease. The process includes inserting a catheter through a patient's radial artery and guiding the catheter to the heart via fluoroscopic imaging. After the procedure, the catheter is removed and a hemostasis device is used to cease bleeding. Current products on the market lack one or more of the following requirements: safely reduce active compression time; keep the patient's wrist hyper-extended to about 45° during surgery to aid in access; restrict the patient's wrist movement to less than 5° after procedure; and guarantee the patency of the radial artery after the procedure.

The device meets all of the above requirements in a cost-effective manner. It may reduce the time clinicians spend checking on patients recovering from surgery. Patients may experience a quicker, more comfortable and safer recovery.

The hemostatic film that is placed on the balloon where it interfaces with the radial artery access site provides additional benefits. As described above, the clear film is used to induce hemostasis and mitigate the risk of infection. Further, the method used to detect patent hemostasis in the radial artery (i.e., the use of a sensor to detect pressure fluctuations in a balloon in order to detect flow through the radial artery) provides additional advantages, as described above.

Many alterations and modifications may be made by those having ordinary skill in the art, given the benefit of present disclosure, without departing from the spirit and scope of the invention. Therefore, it must be understood that the illustrated embodiments have been set forth only for the purposes of example, and that it should not be taken as limiting the invention as defined by the following claims. The following claims, therefore, are to be read to include not only the combination of elements which are literally set forth but all equivalent elements for performing substantially the same function in substantially the same way to obtain substantially the same result. The claims are thus to be understood to include what is specifically illustrated and described above, what is conceptually equivalent, and also what incorporates the essential idea of the invention.

That which is claimed is:

1. A device for fixating and/or supporting a wrist and hand of a patient during a radial artery procedure, the device comprising:
a frame comprising:
a wrist frame member having a wrist support surface to receive the wrist of the patient; and
a hand frame member having a hand support surface to receive the hand of the patient, wherein the hand support member is pivotally connected to the wrist support member;
wherein the hand frame member is movable relative to the wrist frame member from a first position, wherein the hand support surface is at a first angle relative to the wrist support surface to hyperextend the wrist to promote access to the artery for the procedure, and a second position, wherein the hand support surface and the wrist support surface are generally aligned to straighten the wrist and promote hemostasis after the procedure.

2. The device of claim 1, further comprising:
a wrist strap attached to the wrist frame member, the wrist strap configured to overlay the wrist of the patient and urge the wrist of the patient against the wrist support surface; and
a hand strap attached to the hand frame member, the hand strap configured to overlay the hand of the patient and urge the hand of the patient against the hand support surface.

3. The device of claim 2, wherein:
the wrist strap has first and second opposed ends with at least one of the first and second ends removably attached to the wrist frame member; and
the hand strap has first and second opposed ends with at least one of the first and second ends removably attached to the hand frame member.

4. The device of claim 2, further comprising:
an inflatable member strap having first and second opposed ends attached to the wrist frame member; and
an inflatable member disposed between the first and second ends of the inflatable member strap.

5. The device of claim 4, further comprising a pressurized fluid source in fluid communication with the inflatable member and configured to inflate the inflatable member,

6. The device of claim 5, wherein the pressurized fluid source comprises a pump disposed on the wrist frame member.

7. The device of claim 5, further comprising a valve disposed between the pressurized fluid source and the inflatable member, the valve movable between a first position wherein pressurized fluid from the pressurized fluid source flows to the inflatable member to inflate the member and a second position wherein fluid is released from the inflatable member.

8. The device of claim 7, wherein the valve is disposed on the wrist frame member.

9. The device of claim 5, further comprising an instrumentation port in fluid communication with the inflatable member, the instrumentation port configured to connect with instrumentation to monitor and/or display the pressure of the inflatable member.

10. The device of claim 4, wherein at least one of the first and second ends of the inflatable member strap is releasbly attached to the wrist frame member.

11. The device of claim 4, wherein the inflatable member strap comprises first and second straps with the inflatable member disposed therebetween, and wherein at least one of the first and second straps is releasbly attached to the wrist frame member.

12. The device of claim 4, wherein the inflatable member is adjustable along a length of the inflatable member strap.

13. The device of claim 4, wherein a bottom surface of the inflatable member comprises a hemostatic biomaterial.

14. The device of claim 13, wherein the hemostatic biomaterial comprises chitosan.

15. The device of claim 4, further comprising a rigid guard disposed between the first and second ends of the inflatable member strap and above the inflatable member, the guard configured to urge the inflatable member against the wrist of the patient as the inflatable member is inflated.

16. The device of claim 1, further comprising a support member extending between a bottom surface of the wrist frame member and the hand frame member.

17. The device of claim 1, wherein each of the wrist frame member and the hand frame member have an arcuate profile.

18. The device of claim 1, wherein the wrist support surface comprises a pad attached to the wrist frame member and the hand support surface comprises a pad attached to the hand frame member.

19. The device of claim 1, wherein the wrist frame member and the hand frame member are pivotally connected by a pivot mechanism that is configured to lock the frame in each of the first position and the second position.

20. A method for performing catheterization via a radial artery and thereafter establishing hemostasis of the radial artery, the method comprising:
positioning a wrist and an associated hand of a patient in a device such that the posterior aspect of the wrist rests against a support surface of a first portion of the device and such that the dorsal aspect of the hand rests against a support surface of a second portion of the device, wherein the first and second portions are movably coupled together;
pivoting the second portion of the device downwardly to an angled position such that the support surface of the second portion is at an angle of between about 30 and 60 degrees relative to the support surface of the first portion and such that the wrist is hyperextended or supinated;
locking the second portion in the angled position; and
inserting a catheter in the radial artery of the hyperextended wrist.

21. The method of claim 20, further comprising:
removing the catheter from the transradial artery, thereby exposing a puncture site;
pivoting the second portion of the device upwardly to a flat position such that the support surface thereof and the support surface of the first portion are generally aligned;
positioning and securing an inflatable member over the puncture site; and
inflating the inflatable member to a pressure sufficient to seal the radial artery.

22. The method of claim 21, further comprising:
monitoring the pressure of the inflatable member to determine whether the artery is patent or occluded; and
adjusting the pressure of the inflatable member to establish hemostasis of the radial artery.

23. The method of claim 20, further comprising positioning and securing a wrist strap over the anterior aspect of the wrist and positioning and securing a hand strap over the palmar aspect of the hand.
